(19) United States
(12) Patent Application Publication
(10) Pub. No.: US 2005/0003557 A1
(43) Pub. Date: Jan. 6, 2005

(54) Method for immobilizing glycoproteins, constructs and apparatuses made thereby

(75) Inventor: Holger Klapproth, Freiburg (DE)

Correspondence Address: GRIFFIN & SZIPL, PC, SUITE PH-1, 2300 NINTH STREET, SOUTH, ARLINGTON, VA, 22204, US

(73) Assignee: Micronas Holding GmbH, Freiburg (DE)

(21) Appl. No.: 10/793,323

(22) Filed: Mar. 5, 2004

(30) Foreign Application Priority Data
Mar. 6, 2003 (DE) 103 10 193.4

(51) Int. Cl. G01N 33/543

(52) U.S. Cl. 436/518

(57) ABSTRACT

A method for covalently immobilizing glycoproteins such as immunoglobulins on solid, semi-solid or gel-like surfaces, and constructs suitable therefor, as well as surfaces and devices that comprise correspondingly immobilized glycoproteins.

This application claims benefit under 35 U.S.C. 119(a) of German Patent Application No. 10310193.4, filed on Mar. 6, 2003, the entire disclosure of which is hereby incorporated by reference.

### FIELD OF THE INVENTION

The present invention relates generally to the field of biotechnology. Particularly, the invention relates to a method for covalently immobilizing glycoproteins, such as immunoglobulins on solid, semi-solid or gel-type surfaces, and constructs suitable for the same, as well as surfaces and apparatuses that comprise correspondingly immobilized glycoproteins.

### BACKGROUND OF THE INVENTION

The scientific and commercial fields of use for surfaces with functionally available glycoproteins or functional fragments of the same are numerous and require a constant optimization of the basic technology in view of increasing performance and quality demands on the apparatuses provided with these surfaces. A distinction is made between covalent and non-covalent coupling in the immobilization of proteins on a substantially solid surface. Whereas the adsorption (take up) of molecules on a surface (for example, polystyrene) takes place by means of intermolecular attraction forces (van der Waals forces), “actual” chemical bonding through formation of common electron pairs (covalent) or stoichiometric charge differences (ionic).

In the immobilization of immunoglobulins such as specific antibodies, conditions are achieved so that at least a bond by interaction effects (hydrophobic) take place. In the alternative, methods which utilize appropriate cross-linking materials can be used if, for the desired application, the advantages of a durable and covalent coupling must be realized. Such methods, as well as cross-linkers suitable for such methods, are known to those with skill in the art and count as being established (for example, G. Hammenson, “Bioconjugate Techniques,” Academic Press (1996)).

The state of the art known cross-linker methods presently have grave disadvantages, through which the quality of correspondingly produced surfaces and devices are negatively affected with respect to specificity and sensitivity. For example, covalent coupling between cross-linkers and proteins takes place by means of active coupling groups that are no longer available for investigations relating to, for example, any following experiments based on the specific binding between proteins (receptors) and binding partners (ligands). Furthermore, known methods, because of a lack of selectivity of the activated coupling groups, allow several cross-linker molecules to be bound to a protein and the correspondingly-produced constructs react therewith and therefore cannot allow them to be used in the below analyses. A further disadvantage resides in the fact that the protein binds directly to the surface by means of its activated coupling groups and the below investigations fail due to sterical problems. In connection with glycoproteins such as antibodies in particular, these drawbacks mean that for a specific receptor/ligand interaction, the necessary binding domains of the epitope of the protein are either not available or not available in a reproducible manner.

An object of the present invention is to produce alternative constructs and methods for immobilizing the same, so that the disadvantages of the state of the art are overcome.

The above object is solved according to the present invention by providing a construct according to the main claim. Particular embodiments of this construct are represented in the dependent claims. In addition, along with the construct, there are provided various surfaces and apparatuses as well as production methods and uses, that are the subject of further claims. Particular embodiments of the same are provided in the corresponding dependent claims.

### SUMMARY OF THE INVENTION

In accordance with the above objects, the present invention provides a construct, comprising: a heterobifunctional cross-linker molecule having a spacer region having a section for non-covalent adsorption onto a surface; and a protein selected from the group consisting of a glycoprotein and a functional fragment of a glycoprotein. According to a further embodiment, the section for non-covalent adsorption is selected from the group consisting of a hydrophobic section, a positively-charged hydrophilic section, and a negatively-charged hydrophilic section. In accordance with yet another embodiment, spacer region comprises a hydrophilic section and a hydrophobic section. Preferably, the cross-linker molecule comprises at least one photoreactive group for covalent coupling with said surface. In another embodiment, the at least one photoreactive group is disposed at a position selected from the group consisting of an end of said spacer, within said spacer, and both within and at an end of said spacer. In a still further embodiment, the at least one photoreactive group is selected from the group consisting of arylazides, benzophenones, benzothiones, anthraquinones, anthrathiones, thymidine, and derivatives thereof. In yet another embodiment, the cross-linker molecule is covalently coupled with said glycoprotein by a carbohydrate component of said glycoprotein. In a further embodiment, the cross-linker molecule is coupled with said functional fragment of the glycoprotein by a thiol group of said glycoprotein.

In yet another embodiment of the present invention, the glycoprotein is an antibody. In a further embodiment, the functional fragment of a glycoprotein is selected from the group consisting of a FAB-fragment, F(ab′)₂ fragment, and a SCAB protein. In a still further embodiment, the glycoprotein or functional fragment thereof is of recombinant origin.

In another embodiment of the present invention, there is provided a surface having a construct according to one of the above embodiments covalently immobilized thereon. The surface may comprising a plurality of constructs. Preferably, the surface comprises a material selected from the group consisting of glass, quartz glass, quartz, silicon, polymers, including PMMA, polystyrene, polyethylene, polypropylene and PVC, membranes including nitrocellulose, nylon and microfibers, and paper.

In a still further embodiment, there is provided an analytical or diagnostic apparatus comprising a construct according to one of the prior embodiments. In a further embodiment, the apparatus has a structure selected from the group consisting of a biochip, a sensor chip, a reaction column and a microtiter plate.

In accordance with a still further embodiment of the present invention, there is provided a method for producing a construct comprising a glycoprotein or functional fragment thereof and a heterobifunctional cross-linker molecule having a spacer region, comprising the following steps: (a) incubating a protein selected from the group consisting of a glycoprotein and a functional fragment thereof under conditions suitable for production of a chemical group selected from the group consisting of aldehyde groups and thiol groups, to produce a product; (b) covalently coupling the product of step (a) with a cross-linker molecule by a chemical group of the cross-linker selected from the group consisting of an amino group and a maleinimide group, to produce a construct; as well as, optionally, (c) purifying or isolating the construct. In another method embodiment, the cross-linker molecule comprises at least one photoreactive group as a second functional group and wherein the spacer region has a section for non-covalent adsorption onto a surface. In a still further method embodiment, the at least one photoreactive group is disposed at a position selected from the group consisting of an end of said spacer, within said spacer, and both within and at an end of said spacer.

A still further embodiment of the present invention provides a construct comprising a glycoprotein or functional fragment thereof and a heterobifunctional cross-linker molecule having a spacer region having a section for non-covalent adsorption on a surface, that is obtainable by the above-described methods. In another embodiment, the glycoprotein is an antibody, and, in other embodiments the functional fragment of the glycoprotein selected from the group consisting of a FAB fragment, F(ab′)₂ fragment, and a SCAB protein.

In a still further embodiment, there is provided a method, comprising the steps of: providing a construct according to one of the above embodiments; and incorporating the construct into an analytical or diagnostic apparatus, wherein the apparatus is selected from the group consisting of a biochip, a sensor chip, a reaction column and a microtitre plate.

The invention also provides a process for covalent immobilization of a glycoprotein or functional fragment thereof on a surface with use of a heterobifunctional cross-linker molecule, comprising the following steps: (a) applying to a surface a construct of a glycoprotein or functional fragment thereof and a heterobifunctional cross-linker molecule having a spacer region and at least one photoreactive group; and (b) immobilizing the construct on the surface by irradiation of a contact point with light of a suitable wavelength.

In further embodiments, the invention provides an analytical or diagnostic device comprising a surface according to the invention, wherein the device preferably has a structure selected from the group consisting of a biochip, a sensor chip, a reaction column and microtiter plate.

Further objects, features and advantages of the invention will become apparent from the Detailed Description of Illustrative Embodiments, which follows.

### DETAILED DESCRIPTION OF ILLUSTRATIVE EMBODIMENTS

The invention will now be described with reference to certain non-limiting, illustrative embodiments.

Numerous vital cell surface proteins and secretory proteins comprise one or more covalently bonded mono and/or oligo-saccharides and are therefore designated glycosylated proteins or glycoproteins. With the exception of bacterial cells, glycoproteins are distributed in nearly all organisms and cell types. Characteristic sugar components of glycoproteins are hexose, galactose, amanose and glucose as well as N-acetylhexosamine (N-acetylgalactosamine, N-acetylglucosamine) and include, as chain members, sialene acid (N-acetylneuramine acids) and fucose. The oligo-saccharide chains are mostly branched and without periodic sequence, and built with, in general, two to ten mono-saccharide units. The binding of mono, or, as the case may be, oligo-saccharides to the protein components in question is carried out O-glycosydically over hydroxyl groups of serine or, as the case may be threonine residues, N-glycocidically over terminal amino groups, co-amino groups of lysine residues, or, as the case may be, amide groups of asparagine residues, and/or estherglycolidic over the carboxyl groups of asparagine and glutamic acid residues. The number of carbohydrate coupling positions, or, as the case may be, carbohydrate component of glycoproteins varies between only one or several hundred. The glycoproteins are significant components of the cell membrane as well as the extracellular matrix (glycosamine-glycan, lectine, cell wall proteins). The glycosylation pattern of glycoproteins has an information character and is therefore used for a tissue- and cell-specific as well as developmentally-dependent construction of cell surfaces. The immune system, viruses and sperm, for example, use these highly specific patterns for recognizing and adsorption onto the target cells. Because of their numerous hydroxylgroups, bound carbohydrates often increase the solubility of proteins.

The structure of N- and O-bonded oligo-saccharides are very different. A common characteristic of all glycoproteins is however the presence of at least one terminal glycosyl residue that can be, under proper conditions, converted to an aldehyde form by oxidation.

The class of glycoproteins encompasses immunoglobulins, associated with formation of lymphocytes, or as the case may be, plasma cells after contact of the organism with an antigen, and are significant as antibodies in serum, intracellular fluid and body secretions that are significant for humoral immunity. Immunoglobulins comprise two paired identical polypeptide chains with a carbohydrate component, the so-called light or L-chain and the so-called heavy or H-chain that are bound to one another by symmetrically disposed disulfide bridges. Whereas the amino terminal, in a variable amino sequence portion of the molecule carries the antigen binding site, the carboxy terminal portion has a relatively constant structure. Various immunoglobulin fragments (functional fragments) result after splitting by proteolytic enzymes. Splitting with papain results in two identical monovalent Fab-fragments (antigen binding fragments without agglutinizing or precipitating properties) and one Fc-fragment (for particular biological functions such as binding with cellular receptors). By splitting with pepsin, there result one divalent, so-called F(ab′)₂-fragment (producing an agglutination or precipitation after binding with antigen) and smaller subcomponents of the Fc-fragment. In a known manner, immunoglobulins can be classified according to physicochemical and biological (physiological and antigenic) properties of the heavy chains into five immunoglobulin classes (IgG, IgM, IgA, IgD, IgE). The light chains are classified into two types (κ and λ) based on their primary structure and antigenic properties, and they are not class specific.

The carbohydrate component of, for example, serum antibody proteins comprise typically N-acetylglucosamine, manose, fucose, galactose and N-acetylneuraminic acid (NANA). The differences between various proteins relate primarily to the number of branches, the number of NANA residues, as well as the chemical nature of the coupling between the N-acetylneuraminic acid and galactose. The carbohydrate portion of antibodies in the region of the heavy chain carries on its outer end at least one NANA residue, which is negatively charged and increases the solubility of the molecule.

The term “glycoprotein” according to the present invention does not encompass merely native molecules, such as those isolated from natural sources, but also derivative forms, fragments and derivatives, as well as recombinant forms and artificial molecules, so far as these have at least the properties of native molecules.

According to one aspect of the present invention, there is provided a construct comprising a glycoprotein or functional fragment of the same and a heterobifunctional cross-linker molecule with a spacer region having a portion for non-convalent adsorption onto a surface.

It has been shown, according to the present invention, that the immobilization of a glycoprotein or functional fragment of the same at a defined discrete position of the surface is significantly facilitated by preparation of a section of the spacer region for non-covalent adsorption.

The portion of the spacer facing the glycoprotein is preferably compatible in its chemical properties with the solution, or, as the case may be, buffer system, in which the subsequent interaction between receptor and ligand is to take place. To the extent that the incubation of the probe to be examined with the immobilized construct takes place in aqueous medium, the spacer can for example comprise polyethyleneglycol or polyethyleneoxide. In this case, the portion of the spacer facing away from the glycoprotein should preferably be so formed, that it cannot react with the first mentioned portion and is further capable of associating with the surface on which immobilization is to take place.

According to a preferred embodiment, the section for non-convalent adsorption is selected, according to the properties of the surface affected, from among hydrophobic, or positive or negatively charged hydrophilic sections. In most practical applications, hydrophobic surfaces (for example, polystyrene) are used, so that the space or section should have predominantly hydrophobic properties. With the use of hydrophilic surfaces, the spacer section should be hydrophilic and have a charge whose polarity is different from that of the surface.

In the case of, for example, a hydrophilic, negatively charged surface, the section of the spacer should comprise polycationic monomers. Should a hydrophilic, positively charged surface be immobilized, the section of the spacer should in contrast be composed of polyanionic monomers.

In accordance with a particularly preferred embodiment, the spacer comprises a hydrophilic and a hydrophobic section. Because most receptor/ligand assays that use antibodies or functional fragments thereof take place in aqueous solution or buffer systems, the hydrophilic portion should be located on the glycoprotein, so that the hydrophobic portion is available for immobilization on the mostly hydrophobic surface. If the construct for immobilization comprising glycoprotein and cross-linker-molecule-containing spacer is applied to the surface, an absorption of the construct on the hydrophobic surface results. Because the hydrophobic section of the spacer tends to adsorb onto the hydrophobic surface, the covalent immobilization of the construct in the application region takes place by means of the cross-linker groups found in the hydrophobic section.

In accordance with a further preferred embodiment, the cross-linker molecule for covalent coupling with the surface comprises at least one photoreactive group, which is found in the terminus of the spacer and/or is an integral component of the spacer and is activated by irradiation with light energy of the preferred wavelength in region of from 260 to 320 nm (photoreactive group). The selection of suitable photoreactive groups is made preferably from among arylazides, benzophenones, benzothiones, anthraquinones, anthrathiones (such as anthracene-9, 10-dione, and thymidine. The incorporation of one or more suitable photoreactive groups, that differ from one another, can be accomplished by means of state of the art methods such as copolymerization (for example, by means of DNA synthesis apparatuses) during the production of the spacers and/or by means of conventional cross-linkers onto the end of the already-fabricated spacer. One such suitable cross-linker is, for example, NHS-ASA (Pierce, Product No. 27714). A preferred spacer according to the present invention can be produced with a typical DNA synthesis apparatus and comprises an amino-linker (usable as a phosphoramidite), followed by 15 units of deoxyinosine (hydrophilic), 15 C₆ spacers (hydrophobic) and three units of deoxythymidine (photoreactive groups).

It is further preferred that the cross-linker molecule is coupled covalently with the carbohydrate component of the glycoprotein.

The reactive groups of the cross-linkers provided for coupling the glycoprotein are selected in accordance with their chemical properties that should result in a coupling reaction on the site of the activatable groups on the site of the glycoprotein, and should be selected so that they are reactive with aldehyde groups. A particularly suitable cross-linker molecule according to the present invention, therefore, includes an amino group so that the coupling of the glycoproteins can result by its activated aldehyde groups. In the case of the desired immobilization of, for example, an antibody fragment, it is preferred that the cross-linker molecule comprises a maleinimide group that can be covalently coupled over the thiol groups of the functional fragment of the glycoprotein.

Furthermore, it is particularly preferred that the glycoprotein or fragment or the fragment of an antibody or, as the case may be, an FAB- or F(ab′)₂ fragment or a SCAB protein, whereby the protein or fragment can be of recombinant origin.

According to a further aspect, the present invention relates to a surface having a construct covalently immobilized thereon as defined above, in accordance with the present invention, whereby the surface is preferably provided with a plurality of constructs according to the present invention for parallel investigations.

The material according to the surface of the present invention is preferably selected from the group consisting of glass, quartz glass, quartz, silicon, polymers including PMMA, polystyrene, polyethylene, polypropylene and PVC, membranes including those of nitrocellulose, nylon and microfibers, and paper, as well as combinations of the same.

According to a further aspect the present invention there is provided an analytical or diagnostic apparatus, comprising a construct according to the present invention or a surface of the above defined kind, whereby the apparatus is in the form of a biochip, sensor chip, reaction column, or a microtiter plate. It follows from the above that the present invention further comprises a method for production of a construct comprising a glycoprotein or functional fragment of the same and a heterobifunctional cross-linker molecule with a spacer region, comprising the following steps:
(a) incubation of a glycoprotein or functional fragment of the same under suitable conditions for producing aldehyde, or, as the case may be, thiol groups;
(b) covalent bonding of the product of step (a) with a cross-linker molecule by means of the cross-linker molecule's amino, or, as the case may be, maleinimide groups; as well as optionally
(c) cleaning and or isolating the construct.

According to the present invention, it has been discovered that glycoproteins such as, particularly immunoglobulins (antibodies) can be immobilized in a defined orientation on surfaces, wherein the at least one glycosyl residue of the carbohydrate component is converted to an aldehyde under oxidative conditions and the subsequent immobilization results through the modified molecule on an amino-modified surface. In this manner, it is ensured that the binding domains of the covalently bonded immunoglobulins are available for medical and diagnostic applications of the desired interaction between receptor and ligand.

In accordance with a preferred embodiment, the heterobifunctional cross-linker molecule contains, as a second functional group in addition to the amino or maleinimide group, at least one reactive group, and the spacer region comprises a section for non-covalent adsorption onto a surface, whereby with respect to these features reference is made to the above embodiments. It is furthermore preferred that the at least one photoreactive group is disposed at the terminus of the spacer and/or is an integral component of the spacer.

The present invention relates in the above manner to constructs that comprise a glycoprotein or functional fragment of the same and a heterobifunctional cross-linker molecule with a spacer region, which spacer region has a section for non-covalent adsorption onto a surface (see above), and is obtainable by means of the above method according to the present invention. Even in these constructs according to the present invention, it is preferred that the glycoprotein or fragment is an antibody, or as the case may be an FAB- or F(ab′)₂ fragment or a SCAB protein, whereby the protein or fragment can be of recombinant origin.

According to a further aspect, the present invention relates to the use of a construct according to the present invention or a surface according to the present invention for production of an analytical or diagnostic device, preferably in the form of a biochip, sensor chip, reaction column or a microtiter plate. The present invention further relates to a method for covalently immobilizing a glycoprotein or functional fragment thereof on a surface by use of a heterobifunctional cross-linker molecule, comprising the following steps:
(a) applying to a surface a construct of a glycoprotein or functional fragment thereof and a heterobifunctional cross-linker molecule with a spacer region and at least one photoreactive group; and
(b) immobilizing the construct on the surface by irradiation of the contact region with light of a suitable wavelength.

In a preferred embodiment, a construct according to the present invention of the above defined kind is used in step (a).

Lastly, the present invention relates to the use of the construct of the present invention, the surface according to the present invention, or the device according to the present invention in the framework of an analytical or diagnostic device.

While the invention has been described with reference to certain illustrative embodiments, one of ordinary skill in the art will recognize that additions, deletions, substitutions, modifications and improvements can be made while remaining within the scope and spirit of the present invention as defined by the appended claims.

1. A construct, comprising:
a heterobifunctional cross-linker molecule having a spacer region having a section for non-covalent adsorption onto a surface; and
a protein selected from the group consisting of a glycoprotein and a functional fragment of a glycoprotein.

2. A construct according to claim 1, wherein said section for non-covalent adsorption is selected from the group consisting of a hydrophobic section, a positively-charged hydrophilic section, and a negatively-charged hydrophilic section.

3. A construct according to claim 2, wherein said spacer region comprises a hydrophilic section and a hydrophobic section.

4. A construct according to claim 1, wherein said cross-linker molecule comprises at least one photoreactive group for covalent coupling with said surface.

5. A construct according to claim 4, wherein said at least one photoreactive group is disposed at a position selected from the group consisting of an end of said spacer, within said spacer, and both within and at an end of said spacer.

6. A construct according to claim 4, wherein said at least one photoreactive group is selected from the group consisting of arylazides, benzophenones, benzothiones, anthraquinones, anthrathiones, thymidine, and derivatives thereof.

7. A construct according to claim 1, wherein said cross-linker molecule is covalently coupled with said glycoprotein by a carbohydrate component of said glycoprotein.

8. A construct according to claim 1, wherein said cross-linker molecule is coupled with said functional fragment of the glycoprotein by a thiol group of said glycoprotein.

9. A construct according to claim 1, wherein said glycoprotein is an antibody.

10. A construct according to claim 1, wherein said functional fragment of a glycoprotein is selected from the group consisting of a FAB-fragment, F(ab′)₂ fragment, and a SCAB protein.

11. A construct according to claim 1, wherein said glycoprotein or functional fragment thereof is of recombinant origin.

12. A surface having a construct according to claims 1 covalently immobilized thereon.

13. A surface according to claim 12, comprising a plurality of constructs.

14. A surface according to claim 12, wherein the surface comprises a material selected from the group consisting of glass, quartz glass, quartz, silicon, polymers, including PMMA, polystyrene, polyethylene, polypropylene and PVC, membranes including nitrocellulose, nylon and microfibers, and paper.

15. An analytical or diagnostic apparatus comprising a construct according to claim 1.

16. An analytical or diagnostic device according to claim 15 having a structure selected from the group consisting of a biochip, a sensor chip, a reaction column and a microtiter plate.

17. A method for producing a construct comprising a glycoprotein or functional fragment thereof and a heterobifunctional cross-linker molecule having a spacer region, comprising the following steps:
(a) incubating a protein selected from the group consisting of a glycoprotein and a functional fragment thereof under conditions suitable for production of a chemical group selected from the group consisting of aldehyde groups and thiol groups, to produce a product;
(b) covalently coupling the product of step (a) with a cross-linker molecule by a chemical group of the cross-linker selected from the group consisting of an amino group and a maleinimide group, to produce a construct; as well as, optionally,
(c) purifying or isolating the construct.

18. A method according to claim 17, wherein said cross-linker molecule comprises at least one photoreactive group as a second functional group and wherein the spacer region has a section for non-covalent adsorption onto a surface.

19. A method according to claim 18, wherein said at least one photoreactive group is disposed at a position selected from the group consisting of an end of said spacer, within said spacer, and both within and at an end of said spacer.

20. A construct comprising a glycoprotein or functional fragment thereof and a heterobifunctional cross-linker molecule having a spacer region having a section for non-covalent adsorption on a surface, obtainable by the method of claim 17.

21. A construct according to claim 20, characterized in that the glycoprotein is an antibody.

22. A construct according to claim 20, characterized in that the functional fragment of the glycoprotein selected from the group consisting of a FAB fragment, F(ab′)₂ fragment, and a SCAB protein.

23. A method, comprising the steps of:
providing a construct according to claim 20; and
incorporating the construct into an analytical or diagnostic apparatus, wherein the apparatus is selected from the group consisting of a biochip, a sensor chip, a reaction column and a microtitre plate.

24. A process for covalent immobilization of a glycoprotein or functional fragment thereof on a surface with use of a heterobifunctional cross-linker molecule, comprising the following steps:
(a) applying to a surface a construct of a glycoprotein or functional fragment thereof and a heterobifunctional cross-linker molecule having a spacer region and at least one photoreactive group; and
(b) immobilizing the construct on the surface by irradiation of a contact point with light of a suitable wavelength.

25. A method according to claim 24, wherein the construct is a construct according to claim 1.

26. A method according to claim 24, wherein the construct is a construct according to claim 20.

27. An analytical or diagnostic device comprising a surface according to claim 12.

28. An analytical or diagnostic device according to claim 17 having a structure selected from the group consisting of a biochip, a sensor chip, a reaction column and microtiter plate.
